(19) United States
(12) Patent Application Publication
(10) Pub. No.: US 2006/0122581 A1
(43) Pub. Date: Jun. 8, 2006

(54) Multiple energy delivery device

(76) Inventor: Moshe Ein-Gal, Ramat Hasharon (IL)

Correspondence Address: Dekel Patent Ltd.;Beit HaRofim, Room 27, 18 Menuha VeNahala Street, Rehovot, IL

(21) Appl. No.: 10/983,579

(22) Filed: Nov. 9, 2004

(51) Int. Cl. A61B 18/18

(52) U.S. Cl. 606/2

(57) ABSTRACT

A multiple energy delivery device including a plurality of energy conduit groups, each group including at least one energy conduit, wherein a proximal end of each energy conduit group is connected to an energy source (e.g., laser and/or RF energy source), and distal ends of the energy conduit groups are mounted in a common guide member, wherein outputs of the energy sources are deliverable through a distal portion of the guide member.

### FIELD OF THE INVENTION

This invention relates generally to energy delivery devices, such as RF energy devices and optical fiber laser devices and particularly to a device that enables delivery of energy from multiple energy sources.

### BACKGROUND OF THE INVENTION

Laser devices are used in many medical and dental applications, such as but not limited to, incision, necrosis or killing of live tissue, excision or removal of tissue and structure, and cauterization of tissue.

For example, skin problems are pervasive in society. People suffer from conditions ranging from the cosmetic, such as benign discoloration, to fatal ailments, such as malignant melanomas. Treatments range from cosmetic to surgical excision. Removal of the epidermis may eliminate superficial sun damage, including keratoses, lentigenes, and fine wrinkling. Removal of the most superficial portions of the dermis, i.e. the uppermost papillary dermis, may eliminate solar elastosis and ameliorates wrinkling, with little or no scarring.

One treatment uses a short pulse carbon dioxide laser to coagulate a layer of skin to a depth of about 50-100 μm/pulse. This treatment is sometimes referred to as a “laser peel.” CO₂ laser radiation (in the 9-11 μm region of the infrared) is strongly absorbed by water (contained in all tissue). When the energy/unit volume absorbed by the tissue is sufficient to vaporize the water, a microscopically thin layer of tissue at the surface of the irradiated region is rendered necrotic. After irradiation, the dehydrated, necrotic surface layer is mechanically removed, and additional irradiation takes place, this process being repeated until the desired depth of tissue is removed. In medical terms, tissue is removed with less collateral damage than with other modalities, e.g., liquid nitrogen, cautery, chemical peels.

Another treatment uses a pulsed erbium YAG (Er:YAG) laser, emitting radiation at 2.94 μm in the infrared, where water absorption is even stronger than at CO₂ wavelengths. Er:YAG light is approximately 10 times more strongly absorbed in skin than CO₂ laser light. Ultraviolet wavelength lasers have also been used. Examples of the above are discussed in U.S. Pat. No. 6,447,503.

Another well-known medical use of lasers is in the field of transmyocardial revascularization (TMR), which involves the creation of pathways or channels into the myocardium. This may be performed surgically from an outer epicardial surface of the heart or from an inner endothelium cell covered surface of the endocardium chamber in a percutaneous transluminal myocardial revascularization (PTMR). As described in such patents as U.S. Pat. No. 59,318,341, lasers may be used to create channels in the myocardium, such as with a holmium:YAG laser source operating at around 5 Hz with a laser pulse width between 100-250 microseconds, and approximately 0.7-0.9 Joules/mm².

The use of lasers to create the channels must be carefully controlled so as not to cause arrhythmia. Arrhythmia is a disturbed heart rhythm that often takes over as the primary rhythm of the heart, as evidenced by a rapid flutter or other rhythm of the heart muscle, which renders it ineffective at pumping blood through the vasculature. The process of delivering laser energy to tissue results in polarization of individual cells of the heart in the area of delivery of the laser energy. Polarization of the specialized conducting cells as well as myocardial cells drives the action potential of cells resulting in responsive contractile motion. Delivering laser energy can disrupt the normal rhythm of the heartbeat since the cardiac rhythm can be side-tracked to that of the polarized cells as opposed to propagating through the heart along the normal path of the impulse.

Another family of uses for medical lasers as described in such patents as U.S. Pat. No. 5,860,972, includes urological procedures. Such procedures include lithotripsy (e.g., destruction of urinary tract calculi), performing partial or full nephrectomies (removal of one or both of the kidneys), laser-assisted trans-urethral resections of the prostate (TURP) for managing benign prostatic hyperplasia (BPH), treatment of superficial bladder carcinomas or tumors, and other laparascopic procedures. For example, lithotripsy is most commonly achieved by having the laser fiber in contact with the stone.

A problem encountered in many medical laser applications is the difficulty or inability to deliver a large amount of energy over a single fiber or few optical fibers.

Trimedyne, Inc. of Irvine, Calif., manufactures a laser commercially available as the Trimedyne laser (1210-VHP), which attempts to provide more power over a single fiber. It is a Holmium:YAG laser that develops a double pulse and works by generating both heat and acoustic shock to disintegrate calculi.

Other devices are known for combining power from more than one laser or RF source. U.S. Pat. No. 6,652,516 to Gough describes cell necrosis apparatus that has a flexible introducer including a lumen and a distal end sufficiently sharp to penetrate tissue. An energy delivery device is positionable in the introducer as the introducer is advanced through tissue. The energy delivery device includes a first RF electrode with a tissue piercing distal portion and a second RF electrode with a tissue piercing distal portion. An electromagnetic energy source is configured to be coupled to the cell necrosis apparatus with one or more cables. The electromagnetic energy source can be an RF source, microwave source, short wave source, laser source and the like. The cell necrosis apparatus can include energy delivery devices that are RF electrodes, microwave antennas, as well as combinations thereof. The electromagnetic energy source may be a combination RF/microwave box. Further a laser optical fiber, coupled to a laser source can be introduced through the introducer and/or energy delivery device. The introducer and/or a secondary electrode can be an arm for the purposes of introducing the optical fiber.

### SUMMARY OF THE INVENTION

The present invention seeks to provide a novel multiple energy delivery device, which enables delivery of energy from multiple energy sources, as is described more in detail hereinbelow.

There is thus provided in accordance with an embodiment of the present invention a multiple energy delivery device including a plurality of energy conduit groups, each group including at least one energy conduit, wherein a proximal portion of each energy conduit group is connected to an energy source, and distal portions of the energy conduit groups are mounted in a common guide member, wherein outputs of the energy sources are deliverable through the guide member.

In accordance with an embodiment of the present invention the distal portions of the energy conduit groups may be adjacent and abut one another in the guide member. Alternatively, they may be spaced from one another in the guide member.

Further in accordance with an embodiment of the present invention the guide member orients the distal portions of the energy conduit groups to point in a predetermined direction, e.g., to a focal point.

Any number of the energy conduit groups may be connected to different energy sources. A controller may be operationally connected to the energy sources and operative to control operational parameters of the energy sources. The energy sources may be operative to deliver energy capable of performing incision, necrosis, excision, removal and/or cauterization of tissue.

### BRIEF DESCRIPTION OF THE DRAWINGS

The present invention will be understood and appreciated more fully from the following detailed description, taken in conjunction with the drawings in which:

FIG. 1 is a simplified pictorial illustration of a multiple energy delivery device, constructed and operative in accordance with an embodiment of the present invention;

FIG. 2 is a simplified illustration of distal portions of energy conduits of the multiple energy delivery device of FIG. 1 adjacent one another in a guide member, in accordance with an embodiment of the present invention;

FIG. 3 is a simplified illustration of distal portions of energy conduits of the multiple energy delivery device of FIG. 1 spaced from one another in the guide member, in accordance with another embodiment of the present invention; and

FIG. 4 is a simplified illustration of renewing tips of energy conduits and sensing delivered energy, in accordance with an embodiment of the present invention.

### DETAILED DESCRIPTION OF EMBODIMENTS

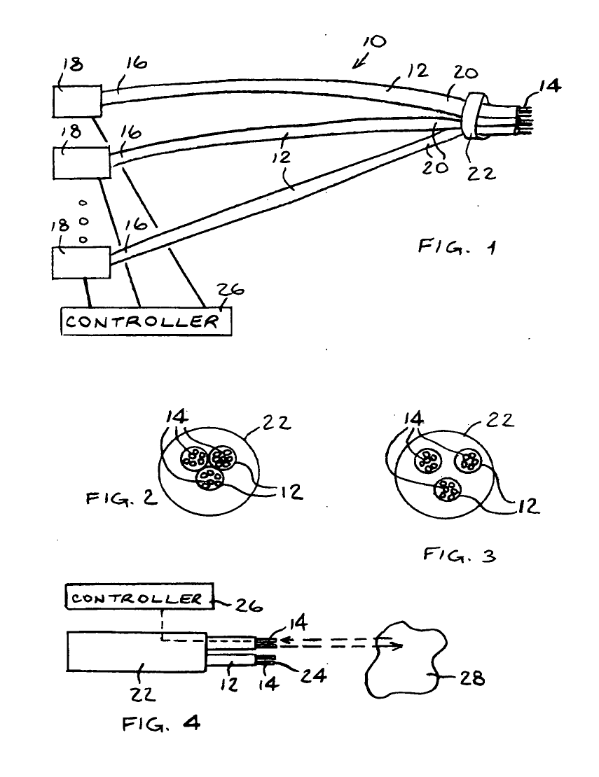

Reference is now made to FIG. 1, which illustrates a multiple energy delivery device 10, constructed and operative in accordance with an embodiment of the present invention.

Multiple energy delivery device 10 may include a plurality of energy conduit groups 12, such as but not limited to, optical fiber groups or RF waveguides. Each energy conduit group 12 may include one or more energy conduits 14, such as but not limited to, optical fibers or miniature waveguides. A proximal end 16 of each energy conduit group 12 may be connected to an energy source 18, such as but not limited to, a laser source or RF source. The distal ends 20 of the energy conduit groups 12 may be mounted in a common guide member 22. Outputs of the energy sources 18 are deliverable through a distal portion of the guide member 22.

Guide member 22 may be made of any material suitable for holding or orienting the energy conduit groups 12, such as but not limited to, plastic or metal. Guide member 22 may orient the distal portions of the energy conduit groups 12 to point in a predetermined direction, such as focusing them to a focal point. As seen in FIG. 2, the distal portions of the energy conduit groups 12 may be adjacent and abut one another in guide member 22. Alternatively, as seen in FIG. 3, the distal portions of the energy conduit groups 12 may be spaced from one another in guide member 22, such as, without limitation, by means of spacers or grooves.

At least two (or every one of the) energy conduit groups 12 may be connected to different energy sources 18 of any kind.

A controller 26 may be operationally connected to the energy sources 18. The controller 26 may control operational parameters of the energy sources 18, such as but not limited to, delivery duration (e.g., pulse duration), power level, wavelength and the like.

The energy delivered through the guide member 22 may be used to perform a variety of procedures, such as but not limited to, incision, necrosis, excision, removal and cauterization of tissue.

Delivery of energy from the multiple energy delivery device 10 generally generates heat, and it is possible that tips of the energy conduits 14 may burn or char after repeated use. As seen in FIG. 4, guide member 22 enables “chopping”, severing or cutting (or equivalent, the terms being used interchangeably throughout) tips 24 of the energy conduits 14 so as to “renew” the tips 24 so as to maintain the intensity and directionality of the power delivered from the tips 24.

As seen in FIG. 4, one or more of the conduits 14, or at least one additional energy conduit 14, may be disposed in guide member 22 to sense the energy or power delivered to a target or focal volume 28 (e.g., by reflection) and transmit the sensed information or signal to a processor, such as but not necessarily, controller 26.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A multiple energy delivery device comprising:
a plurality of energy conduit groups, each group comprising at least one energy conduit, wherein a proximal portion of each energy conduit group is connected to an energy source, and distal portions of the energy conduit groups are mounted in a common guide member, wherein outputs of said energy sources are deliverable through said guide member.

2. The multiple energy delivery device according to claim 1, wherein the distal portions of the energy conduit groups are adjacent and abutting one another in said guide member.

3. The multiple energy delivery device according to claim 1, wherein the distal portions of the energy conduit groups are spaced from one another in said guide member.

4. The multiple energy delivery device according to claim 1, wherein said guide member orients the distal portions of the energy conduit groups to point in a predetermined direction.

5. The multiple energy delivery device according to claim 1, wherein said guide member focus outputs of the energy conduit groups to a focal point.

6. The multiple energy delivery device according to claim 1, wherein at least two energy conduit groups are connected to different energy sources.

7. The multiple energy delivery device according to claim 1, wherein each energy conduit group is connected to a different energy source.

8. The multiple energy delivery device according to claim 6, further comprising a controller operationally connected to said energy sources and operative to control operational parameters of said energy sources.

9. The multiple energy delivery device according to claim 1, wherein said energy sources comprise at least one of laser sources and RF energy sources.

10. The multiple energy delivery device according to claim 1, wherein tips of the energy conduits are cuttable.

11. The multiple energy delivery device according to claim 1, wherein at least one energy conduit is adapted to sense energy delivered by said multiple energy delivery device and transmit sensed information to a processor.

12. The multiple energy delivery device according to claim 1, wherein said energy sources are operative to deliver energy capable of performing at least one of incision, necrosis, excision, removal and cauterization of tissue.
